SEQUENCE LISTING (3 sequences)

SEQ ID NO: 1 - protein, 365 aa, Candida boidinii
MGKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 2 - protein, 367 aa, Candida boidinii
MGKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADELLGYNRERAMKKVEGIYDKILKVFEIAKRDGIPSYLAADRMAEERIEMMRKTRSTFLQDQRNLINFNNK

SEQ ID NO: 3 - protein, 640 aa, Candida boidinii
MASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

(19) United States
(12) Patent Application Publication
(10) Pub. No.: US 2004/0115691 A1
(43) Pub. Date: Jun. 17, 2004

(54) Mutants of enzymes and methods for their use

(76) Inventors: J. David Rozzell, Burbank, CA (US); Ling Hua, Arcadia, CA (US); Martin Mayhew, Arcadia, CA (US); Scott Novick, Santa Clarita, CA (US)

Correspondence Address: CHRISTIE, PARKER & HALE, LLP, P.O. BOX 7068, PASADENA, CA, 91109-7068, US

(21) Appl. No.: 10/617,998

(22) Filed: Jul. 10, 2003

Related U.S. Application Data
(60) Provisional application No. 60/394,886, filed on Jul. 10, 2002.

(51) Int. Cl. C12Q 1/68; C07H 21/04; C12N 9/06

(52) U.S. Cl. 435/6; 435/69.1; 435/191; 435/320.1; 435/325; 536/23.2

(57) ABSTRACT

Mutants of leucine dehydrogenase sequences, formate dehydrogenase sequences and galactose oxidase sequences are provided. An amino acid sequence that is a mutant of a leucine dehydrogenase sequence as described in SEQ ID 2, or its substantial equivalent, contains at least one mutation selected from the group consisting of F102S, V33A, S351T, N145S and like mutations in subsantially equivalent sequences. An amino acid sequence that is a mutant of a formate dehydrogenase sequence as described in SEQ ID 1, or its substantial equivalent, contains at least one mutation selected from the group consisting of D195S, Y196H, K356T and like mutations in subsantially equivalent sequences. An amino acid sequence that is a mutant of a galactose oxidase sequence as described in SEQ ID 3, or its substantial equivalent, contains at least one mutation selected from the group consisting of N25Y, T94A, D216N, R217C, M278T, Y329C, Q406R, Q406L, V492A, V494A, N521S, N535D, T5491, S567T, T578S and like mutations in subsantially equivalent sequences. Deoxyribonucleic acid molecules containing DNA sequences encoding these mutants are also provided.

### CROSS-REFERENCE TO RELATED APPLICATION(S)

This application claims the benefit of U.S. Provisional Patent Application No. 60/394,886; filed Jul. 10, 2002, the entire disclosure of which is incorporated herein by reference.

### FIELD OF THE INVENTION

This invention relates to novel mutants of leucine dehydrogenase, formate dehydrogenase, and galactose oxidase and their applications.

### BACKGROUND

Unnatural or non-proteinogenic amino acids, which are structural analogs of the naturally-occurring amino acids that are the constituents of proteins, have important applications as pharmaceutical intermediates. For example, the anti-hypertensives ramipril, enalapril, benazapril, and prinivil are all based on L-homophenylalanine; certain second generation pril analogs are synthesized from p-substituted-L-homophenylalanine. Various β-lactam antibiotics use substituted D-phenylglycine side chains, and newer generation antibiotics are based on aminoadipic acid and other UAAs. The unnatural amino acids L-tert-leucine, L-nor-valine, L-nor-leucine, L-2-amino-5-[1,3]dioxolan-2yl-pentanoic acid, and the like have been used as a precursor in the synthesis of a number of different developmental drugs.

Unnatural amino acids are used almost exclusively as single stereoisomers. Since unnatural amino acids are not natural metabolites, traditional production methods for amino acids based on fermentation cannot generally be used since no metabolic pathways exist for their synthesis. Given the growing importance of unnatural amino acids as pharmaceutical intermediates, various methods have been developed for their enantiomerically pure preparation. Commonly employed methods include resolutions by diastereomeric crystallization, enzymatic resolution of derivatives, or separation by simulated moving bed (SMB) chiral chromatography. These methods can be used to separate racemic mixtures, but the maximum theoretical yield is only 50%.

In the case of non-proteinogenic alkyl straight-chain and branched-chain amino acids such as L-nor-valine, L-nor-leucine, L-2-amino-5-[1,3]dioxolan-2yl-pentanoic acid, or L-tert-leucine, enzyme-catalyzed reductive amination is an effective method for their synthesis. Whereas the naturally-occurring alkyl and branched-chain amino acids can be produced by fermentation, taking advantage of the existing metabolic pathways to produce these amino acids, stereoselective production of non-proteinogenic analogs and various similar compounds is more difficult. The enzyme leucine dehydrogenase has been shown to be capable of catalyzing the reductive amination of the corresponding 2-ketoacids of alkyl and branched-chain amino acids, and L-tert-leucine has been produced with such an enzyme. Improved rates, activity toward a broader range of substrates, and greater enzyme stability would offer improved biocatalysts for this type of reaction. It is also an object of this invention to describe methods and mutants that can lead to the reductive amination of 2-ketoacids to produce D-amino acids such and the D-counterparts of naturally-occurring amino acids and D-analogs of non-proteinogenic amino acids such as those listed above (D-nor-valine, D-nor-leucine, D-2-amino-5-[1,3]dioxolan-2yl-pentanoic acid, or D-tert-leucine).

Nicotinamide cofactor dependent enzymes are increasingly finding use for the synthesis of chiral compounds. Such processes are now in various stages of scale-up and commercialization. Amino acid dehydrogenases are used industrially to synthesize unnatural L-amino acids such as L-tert-leucine at the multi-ton scale (Scheme 1). (Kragl et al, 1996) Alcohol dehydrogenases have been used to synthesize chiral alcohols, hydroxy esters, hydroxy acids, and amino alcohols. An important feature of these reactions is that they are chiral syntheses, not resolutions, with yields that can approach 100% of theoretical. The starting materials for these types of reactions are the achiral ketones or keto-analogs, which are often readily available at low cost.

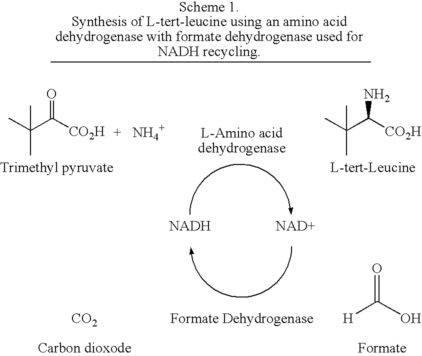

Because of the relatively high cost of nicotinamide cofactors (in comparison to the other starting materials), it is not economically feasible to use the cofactor in stoichiometric quantities. Instead, the cofactor must be regenerated in situ using a suitable recycling system. The recycling method for the commercial production of L-tert-leucine is based on the use of NAD-dependent formate dehydrogenase (FDH) for the regeneration of NADH from NAD+. This is an ideal cofactor recycling system because formate is an inexpensive, water-soluble reductant, the reaction catalyzed by formate dehydrogenase (formate to CO₂) is essentially irreversible, and the only byproduct, carbon dioxide, causes no waste disposal or purification problems. Furthermore, formate dehydrogenase is now available commercially in bulk quantities, as BioCatalytics, Inc. launched the first recombinant form of the enzyme in 2001. The commercial formate dehydrogenase enzyme is, however, specific for NAD+ as its substrate; it shows no activity toward NADP+.

Despite the fact that there is no comparable NADP-utilizing formate dehydrogenase available, there nonetheless exist a number of extremely useful NADP-dependent enzymes. Of particular interest are the NADP-dependent ketoreductases, which catalyze the stereoselective reduction of a broad range of ketones to the corresponding chiral alcohols. In general, the NADP-dependent ketoreductases catalyze reactions on more complex ketones (those that are also more useful synthetically) than the corresponding NAD-dependent enzymes, and ways to exploit their broad catalytic potential are actively being sought. To date, we have used glucose dehydrogenase for NADP+ recycling with some success (Scheme 2). However, there are certain disadvantages to this. Glucose must be fed as the reaction proceeds, and the byproduct, ultimately gluconic acid (from spontaneous hydrolysis of gluconolactone) is produced in equimolar quantities and must be separated from the desired product. The pH will also drop during this process due to gluconolactone hydrolysis, and therefore pH control is necessary. An enzymatic process for the regeneration of NADP+ using formate as depicted in Scheme 3 would thus be strongly preferred.

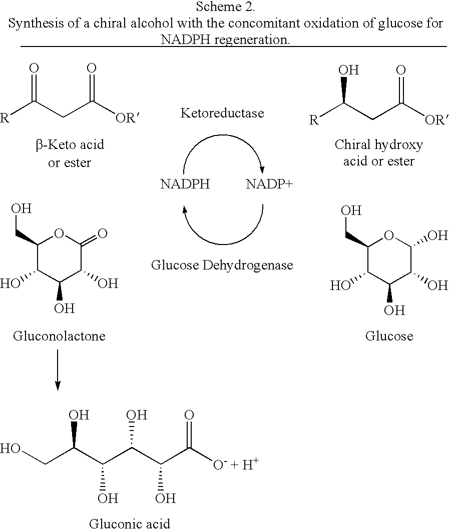

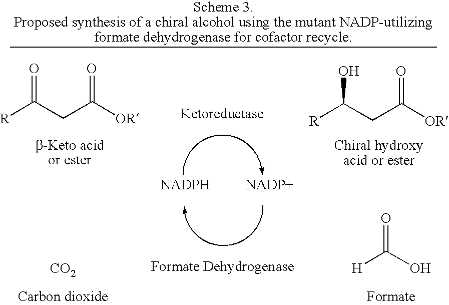

Directed evolution of enzymes is an extremely powerful method to produce new enzymes with specific desired properties. In this technique, the gene encoding the enzyme of interest is mutagenized and transformed into a host strain such as E. coli to produce a library of mutant enzymes. This library, which may contain 5000-20,000 distinct mutants, is screened for an enzyme having the desired property. The mutants that test positive for the screen can then be subjected to further rounds of mutagenesis and screening in an iterative process to obtain an increasingly superior enzyme. This technique has been successfully applied to enhance many properties of enzymes including specific activity, thermostability, substrate specificity, and enantioselectivity.

Similar opportunities exist for the use of inexpensive carbohydrate precursors such as galactose. The enzyme galactose oxidase converts galactose to the corresponding aldehdye at the C-6 position using molecular oxygen as the only co-reactant. Mutants of galactose oxidase that are more active, or that act on other carbohydrate or alcohol starting materials, would be highly desirable catalysts.

### SUMMARY OF THE INVENTION

The present invention is directed to an amino acid sequence that is a mutant of an enzyme selected from the group consisting of leucine dehydrogenase sequences as described in SEQ ID 2, formate dehydrogenase sequence as described in SEQ ID 1, galactose oxidase sequences as described in SEQ ID 3, and substantial equivalents thereof. When the amino acid sequence is a mutant of a leucine dehydrogenase sequence as described in SEQ ID 2 or a substantial equivalent thereof, the amino acid sequence contains at least one mutation selected from the group consisting of F102S, V33A, S351T, N145S and like mutations in subsantially equivalent sequences. When the amino acid sequence is a mutant of a formate dehydrogenase sequence as described in SEQ ID 1 or a substantial equivalent thereof, the amino acid sequence contains at least one mutation selected from the group consisting of D195S, Y196H, K356T and like mutations in subsantially equivalent sequences. When the amino acid sequence is a mutant of a galactose oxidase sequence as described in SEQ ID 3 or a substantial equivalent thereof, the amino acid sequence contains at least one mutation selected from the group consisting of N25Y, T94A, D216N, R217C, M278T, Y329C, Q406R, Q406L, V492A, V494A, N521S, N535D, T5491, S567T, T578S and like mutations in subsantially equivalent sequences.

### DETAILED DESCRIPTION

The present invention is directed toward mutant leucine dehydrogenase enzymes, mutant formate dehydrogenase enzymes, and mutant galactose oxidase enzymes. In one embodiment, the invention is directed to an amino acid sequence that is a mutant of a leucine dehydrogenase sequence as described in SEQ ID 2, or its substantial equivalent, with the amino acid sequence containing at least one mutation selected from the group consisting of F102S, V33A, S351T, N145S and like mutations in substantially equivalent sequences, as well as to a deoxyribonucleic acid molecule containing a DNA sequence encoding the mutated amino acid sequence.

In another embodiment, the invention is directed to an amino acid sequence that is a mutant of a formate dehydrogenase sequence as described in SEQ ID 1, or its substantial equivalent, the amino acid sequence containing at least one mutation selected from the group consisting of D195S, Y196H, K356T and like mutations in substantially equivalent sequences, as well as to a deoxyribonucleic acid molecule containing a DNA sequence encoding the mutated amino acid sequence.

In another embodiment, the invention is directed to an amino acid sequence that is a mutant of a galactose oxidase sequence as described in SEQ ID 3, or its substantial equivalent, said amino acid sequence containing at least one mutation selected from the group consisting of N25Y, T94A, D216N, R217C, M278T, Y329C, Q406R, Q406L, V492A, V494A, N521S, N535D, T5491, S567T, T578S and like mutations in substantially equivalent sequences, as well as to a deoxyribonucleic acid molecule containing a DNA sequence encoding the amino acid sequence.

The invention is also directed to a method for the production of an amino acid that comprises contacting a ketoacid with an amino acid sequence that is a mutant of the leucine dehydrogenase described above in the presence of a reduced nicotinamide cofactor and an ammonia source.

The invention is also directed to a method for recycling a nicotinamide cofactor that comprises contacting an oxidized nicotinamide cofactor with an amino acid sequence that is a mutant of a formate dehydrogenase sequence as described above in the presence of a formate source.

As used herein, the terminology “substantial equivalent” when used to refer to an amino acid or nucleic acid sequence encompasses complementary sequences, derivatives, analogs, homologs and fragments.

A nucleic acid molecule that is complementary to a nucleotide sequence shown or described is one that is sufficiently complementary to the nucleotide sequence shown such that it can hydrogen bond with little or no mismatches to the nucleotide sequences shown, thereby forming a stable duplex. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the amino acid or nucleic acid sequence of the invention can comprise only a portion of the described amino acid or nucleic acid sequence, e.g., a fragment that can be used as a probe or primer or a fragment encoding a biologically active portion. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or amino acid sequences of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence. Alignment can be done manually or using a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. Homologous nucleic acid sequences include those nucleic acid sequences that encode conservative amino acid substitutions (see below) in a polypeptide, as well as a polypeptide having an activity.

The nucleotide sequence determined from the cloning of one gene allows for the generation of probes and primers designed for use in identifying and/or cloning homologues in other cell types, e.g., from other organisms, as well as homologs. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand of the described nucleotide sequence.

Probes based on nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a protein, such as by measuring a level of a nucleic acid in a sample of cells.

The invention further encompasses nucleic acid molecules that differ from the described nucleotide sequences due to degeneracy of the genetic code. These nucleic acids thus encode the same protein as that encoded by the described nucleotide sequence.

Accordingly, in another embodiment, an isolated nucleic acid molecule of the invention is at least 6 nucleotides in length and hybridizes under stringent conditions to the nucleic acid molecule comprising a described nucleotide sequence. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs or other related sequences can be obtained by low, moderate or high stringency hybridization with all or a portion of the particular nucleic acid sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5.degree. C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30.degree. C. for short probes, primers or oligonucleotides (e.g., 10 nt to 50 nt) and at least about 60.degree. C. for longer probes, primers and oligonucleotides. Stringent conditions may also be achieved with the addition of destabilizing agents, such as formamide.

Stringent conditions are known to those skilled in the art and can be found in Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6.times.SSC, 50 mM Tris-HCl (pH 7.5), 1 mM EDTA, 0.02% PVP, 0.02% Ficoll, 0.02% BSA, and 500 mg/ml denatured salmon sperm DNA at 65.degree. C. This hybridization is followed by one or more washes in 0.2.times.SSC, 0.01% BSA at 50.degree. C. An isolated nucleic acid molecule of the invention that hybridizes under stringent conditions to one of the described sequences corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In another embodiment, a nucleic acid sequence that is hybridizable to the nucleic acid molecule comprising a described nucleotide sequence or fragments, analogs or derivatives thereof, under conditions of moderate stringency is provided. A non-limiting example of moderate stringency hybridization conditions are hybridization in 6.times.SSC, 5.times.Denhardt's solution, 0.5% SDS and 100 mg/ml denatured salmon sperm DNA at 55.degree. C., followed by one or more washes in 1.times.SSC, 0.1% SDS at 37.degree. C. Other conditions of moderate stringency that may be used are well known in the art. See, e.g., Ausubel et al. (eds.), 1993, Current Protocols in Molecular Biology, John Wiley & Sons, NY, and Kriegler, 1990, Gene Transfer and Expression, a Laboratory Manual, Stockton Press, NY.

In another embodiment, a nucleic acid that is hybridizable to the nucleic acid molecule comprising a described nucleotide sequence or fragment, analog or derivative thereof, under conditions of low stringency, is provided. A non-limiting example of low stringency hybridization conditions are hybridization in 35% formamide, 5.times.SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40.degree. C., followed by one or more washes in 2.times.SSC, 25 mM Tris-HCl (pH 7.4), 5 mM EDTA, and 0.1% SDS at 50.degree. C. Other conditions of low stringency that may be used are well known in the art (e.g., as employed for cross-species hybridizations). See, e.g., Ausubel et al. (eds.), 1993, Current Transfer and Expression, a Biology, John Wiley & Sons, NY, and Kriegler, 1990, Gene Transfer and Expression, a Laboratory Manual, Stockton Press, NY; Shilo et al., 1981, Proc Natl Acad Sci USA 78: 6789-6792.

In addition to naturally-occurring variants of a given nucleic acid or amino acid sequence that may exist, the skilled artisan will further appreciate that changes can be introduced into a nucleic acid or directly into a polypeptide sequence without significantly altering the functional ability of the protein. In some embodiments, a described nucleotide sequence will be altered, thereby leading to changes in the amino acid sequence of the encoded protein. For example, nucleotide substitutions that result in amino acid substitutions at various “non-essential” amino acid residues can be made in the described sequences. A “non-essential” amino acid residue is a residue that can be altered from the wild-type sequence of without altering the biological activity, whereas an “essential” amino acid residue is required for biological activity. For example, amino acid residues that are conserved among the proteins of the present invention, are predicted to be less amenable to alteration, although some alterations of this type will be possible.

Another aspect of the invention pertains to nucleic acid molecules encoding proteins that contain changes in amino acid residues that are not essential for activity. Such proteins differ in amino acid sequence from the described sequences, yet retain biological activity. In one embodiment, the isolated nucleic acid molecule comprises a nucleotide sequence encoding a protein, wherein the protein comprises an amino acid sequence at least about 45% homologous to a described amino acid sequence. Preferably, the protein encoded by the nucleic acid molecule is at least about 60% homologous to a described sequence, more preferably at least about 70%, 80%, 90%, 95%, 98%, and most preferably at least about 99% homologous to a described sequence.

An isolated nucleic acid molecule encoding a protein homologous to a described protein can be created by introducing one or more nucleotide substitutions, additions or deletions into a described nucleotide sequence such that one or more amino acid substitutions, additions or deletions are introduced into the encoded protein. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a polypeptide is replaced with another amino acid residue from the same side chain family. Alternatively, in another embodiment, mutations can be introduced randomly along all or part of a coding sequence, such as by saturation mutagenesis, and the resultant mutants can be screened for a desired activity to identify mutants that display that desired activity.

As used herein, the terminology “like mutations in substantially equivalent sequences” refers to mutations in substantially equivalent sequences, as defined above, that are in locations different from, but corresponding to, those indicated. For example, deletions or insertions can sometimes occur in a nucleic acid or amino acid sequences, creating substantially equivalent sequences that are “frame-shifted.” These “frame-shifted” sequences maintain a similar or homologous sequence of nucleic acids or amino acids except that the numerical positions of certain individual nucleic acids or amino acids are shifted to a higher number if an insertion of one or more nucleic acids or amino acids has occurred at an earlier point in the sequence. Similarly, the numerical positions of certain individual nucleic acids or amino acids are shifted to a lower number if a deletion of one or more nucleic acids or amino acids has occurred at an earlier point in the sequence.

As a starting point for the evolving of NADP+ accepting FDH, FDH genes are prepared using redesign and synthesis methodology. The gene encoding FDH form Candida boidini has been redesigned and synthesized to enhance its expression in E. coli. The synthesized gene expresses at 20% to 40% of the total protein in the cell, all of which is soluble, active enzyme, resulting in formate dehydrogenase. The high level expression of this gene in functionally active form enables greater sensitivity in the detection of mutants able to accept NADP+ as a substrate.

Mutagenesis libraries of these genes are prepared using methods developed to create mutant genes. Our initial approach focuses on the use of error-prone PCR, such as by error-prone PCR protocol described in detail below. This method has been applied to the directed evolution of other enzymes, including aminotransferases and alcohol oxidases. We have used these methods previously for the generation of mutants of other genes in the successful directed evolution of enzyme activities. The method can be fine-tuned as necessary for mutagenizing the FDH gene. The mutagenized genes generated as described below are transformed into E. coli strain LMG194 or similar for expression and screening.

As a starting point, the template used is the synthetic FDH gene that contains the mutation as described by Gul-Karaguler (2001). This gene, designed especially for high-level expression in E. coli, is subjected to mutagenesis by error-prone PCR according to a modification of the method of May and Arnold [May and Arnold, 2000]. The use of the synthetic gene enhances the success of the mutant library by predisposing all derivative genes for higher expression in our E. coli host. The error-prone PCR is performed in a 100 μL reaction mixture containing 0.25 ng of plasmid DNA as template dissolved in PCR buffer (10 mM Tris, 1.5 mM MgCl₂, 50 mM KCl, pH 8.3), and also containing 0.2 mM of each dNTP, 50 pmol of each primer and 2.5 units of Taq polymerase (Roche Diagnostics, Indianapolis, Ind. USA). The baseline conditions, which can be fine-tuned as necessary, for carrying out the PCR are as follows: 2 minutes at 94° C.; 30 cycles of 30 seconds 94° C., 30 seconds 55° C.; 2 minutes at 72° C. The PCR product is double digested with Nco I and Bgl II and subcloned into pBAD/HisA vector (Invitrogen, Carlsbad, Calif. USA) that has been digested with same restriction enzymes. The resulting mutant library is transformed into an E. coli host strain LMG194 (Invitrogen, Carlsbad, Calif. USA) and plated on LB agar supplied with 100 microgram/mL ampicillin. Individual transformants containing putative mutations are picked into 96-well microtiter plates (hereafter referred to as master plates) containing 0.2 mL LB Broth with 100 microgram/mL ampicillin, and growth is allowed to take place for 8-16 hours at 37° C. with shaking at 200 rpm. Each well in each 96-well master plate is then re-inoculated by a replica plating technique into a new second stage 96-well plate pre-loaded with the same growth media plus 2 g/L of arabinose, and growth is allowed to continue for 5-10 hours at 37° C. with shaking at 200 rpm. The second stage 96-well plates are then centrifuged at 4,000 rpm for 10 minutes, and the supernatant is decanted. The cell pellet in each well is washed with 200 μL of water. The washed cell pellet is suspended in 30 μL of B-Per Bacterial Protein Extraction Reagent (Pierce, Rockford, Ill. USA). Assays are conducted using the reduction of NADP+ in the presence formate as an indicator of activity. The inventors have found that mutagenesis conditions that produce approximately a 30% kill rate (30% of the transformants have inactive enzyme caused by mutations, as assayed against the natural substrates) generate 1-3 mutations per gene, and that this rate of mutagenesis is useful for creating mutant enzymes with modified activities.

After the mutants are generated as described above, colonies are picked robotically using a colony picker (Autogen, Framingham, Mass. USA). Up to approximately 2700 candidate clones can be picked per hour using this colony picker into 96-well (or 384-well) microtiter plates.

Screening is accomplished using a two-stage plating procedure described below for 96-well plates, but which can be adapted to 384-well plates to increase throughput. Each well in each 96-well master mutagenesis plate is re-inoculated by a replica plating technique into a new second stage 96-well plate pre-loaded with the same growth media plus 2 g/L of arabinose. Growth is continued for 5-10 hours at 37° C. with shaking at 200 rpm. After centrifugation at 4,000 rpm for 10 minutes, the supernatant is decanted, and the cell pellets in the second stage 96-well plates are washed with 200 μL of water. The washed cell pellets are then suspended in 30 μL of B-Per Bacterial Protein Extraction Reagent (Pierce, Rockford, Ill. USA) for cell lysis.

After mixing, the suspension of cells in B-Per reagent are allowed to stand for 10 minutes at room temperature. Then, a solution having the following composition is added to each well in the plate using a multi-channel pipetting device:

7.5 μL of a pH 8.0 solution containing 8 mg/mL of NADP+

7.5 μL of a pH 8.0 solution containing 0.25 M ammonium formate

155 μL of 1 mM potassium phosphate buffer, pH 8.0

1.5 μL of a 4 mg/mL solution of bromothymol blue indicator

Wells in which the color changes from blue initially to yellow contain enzymes that are able to oxidize formate with NADP+ as a cofactor. These wells are correlated to the original wells in the master plates to obtain the original clones of FDH. The sensitivity of the method permits the detection of new mutant enzymes having as little as 0.001 micromole per minute per milligram of protein, or about 1/1000^{th} the activity of the enzyme on NAD+.

Background can be reduced by pelleting the cell debris formed by the cell lysis procedure, further enhancing the sensitivity of the screen. This additional step is preferably implemented only if necessary, as it adds an additional centrifugation operation to the overall protocol.

The best mutants from the first round of mutagenesis described above are reconfirmed by assay and then sequenced. The mutation or mutations responsible for increased activity are determined. Combinations of all different mutations that give rise to increased activity for the reduction of NADP+ are prepared and tested to look for synergistic effects of multiple mutations in the gene. The best mutants from screening and from the preparation of new combinations of synergistic mutations are subjected to further rounds of mutagenesis and screening as described above. The further rounds of mutagenesis and screening are carried out iteratively to evolve increasingly superior NADP-utilizing FDH enzymes. In general, the best 3-5 mutants from each round are carried forward into the subsequent round of mutagenesis and screening.

The mutants showing the highest activity from the first and subsequent rounds of mutagenesis and screening are reconfirmed by growing cells containing the gene in multiple 1 liter shake flasks. After growth, the cells are harvested, lysed, and the enzyme is purified via chromatographic (DE or CM cellulose, or other media) or precipitation (heat treatment or ammonium sulfate) methods. SDS-PAGE gels of the crude and purified mutant(s) are taken. Kinetic parameters, V_{Max}, K_{M} (for both formate and NADP+), Kₚ for NADPH, and pH optimum aer determined. The kinetic parameters are preferably determined in two sets of experiments. To determine the kinetic parameters of formate, the mutants aer assayed against various concentrations of formate (0-100 mM) at a high NADP+ concentration (1 mM). The data is fit to the standard Michaelis-Menten equation using nonlinear regression:

$${v}_{i} = \frac{{V}_{Max} {S}_{0}}{{K}_{M} + {S}_{0}}$$

The kinetic values for NADP+ aer determined in a similar way. The activity is measured at various NADP+ concentrations (0-1 mM) at a high formate concentration (50 mM). Since the cofactor product, NADPH, is known to inhibit FDH, the Kₚ can also be determined. For this, the formate and NADP+ concentrations aer fixed at 50 and 0.5 mM, and the NADPH concentration is varied (0-1 mM). The data is fit, using nonlinear regression, to the Michaels-Menten equation modified for product inhibition:

$${v}_{i} = \frac{{V}_{Max} {S}_{0}}{{S}_{0} + {K}_{M} ( 1 + \frac{{P}_{0}}{{K}_{p}} )}$$

Stability of the new enzymes is measured by incubating the mutant FDH(s) in buffer at various temperatures and periodically assaying the enzyme for activity. Stability experiments are carried out for 2 half-lives or 1 month, which ever occurs first.

To demonstrate the applicability of the mutant NADP+ accepting FDH, it is used to synthesize, on the gram scale, a β-hydroxy acid or ester. Initially the synthesis of ethyl 4-chloro-3-hydroxy butyrate from ethyl 4-chloro-acetoacetate is examined. This is a key intermediate in the synthesis of Lipitor™, with demand exceeding 100 tons per year. The inventors have already established that KRED 1007, one of the novel ketoreductases cloned by BioCatalytics, can catalyze the stereoselective reduction of the ketone to produce the S-alcohol, which after displacement of chloride by cyanide, is the correct stereochemistry of the key C-5 intermediate for further conversion into Lipitor™. The reaction sequence to be used is shown in Scheme 4. The net reaction is 4-chloro-acetoacetic ester+formate→optically-pure S-4-chloro-3-hydroxybutyrate ethyl ester.

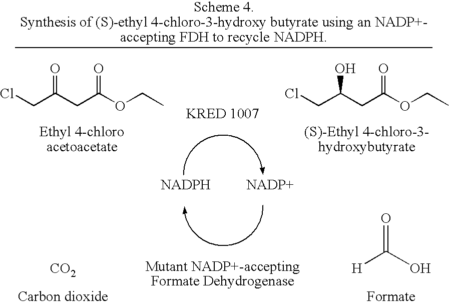

The procedure used is similar to the biphasic system described by Shimizu et at (1990). The substrate and product degrade in water, and therefore a biphasic system is necessary as the substrate and product will partition into the organic phase. To 100 ml of n-butyl acetate, 6 ml of the ethyl 4-chloro-acetoacetate is added. The enzymes, the mutant FDH and a ketoreductase capable of reducing the 2-keto acid to the S-alcohol (BioCatalytics' KRED1007), are added to the aqueous phase (pH 7) to give a total of about 1000 Units each, along with NADP+ and formate at 0.15 and 600 mM each, respectively. The two phases are mixed thoroughly and the progress of the reaction is monitored via gas chromatography. After 100% conversion is obtained, any product in the aqueous phase is extracted into ethyl acetate and combined with the butyl acetate phase. The solvent is removed via rotary evaporation. Product yield, purity, enantiomeric excess, and total turnover of cofactor aer determined. The parameters given above are the starting point and can be adjusted as necessary.

### EXAMPLES

The invention will now be described by the following examples, which are presented here for illustrative purposes and are not intended to limit the scope of the invention.

Materials and Sources:

DNA taq polymerase and T4 DNA ligase can be purchased from Roche Molecular Biochemicals (Branchburg, N.J.). Restriction endonucleases can be obtained from New England Biolabs. The pET15b expression vector and E. coli BL21(DE3) were provided previously by Donald Nierlich (UCLA, Calif.). The pBAD expression vector and E. coli LMG 194 can be purchased from Invitrogen Corporation (Carlsbad, Calif.). The cloning vectors pGEM-3Z, pGEM-5Zf(+) and the host strain E. coli JM109 can be purchased from Promega (Madison, Wis.). Oligonucleotides used for PCR amplification can be synthesized by IDT Inc. (Coralville, Iowa USA) or the University of Florida Core Laboratory (Gainesville, Fla. USA). QIAquick gel extraction kit and QIAprep spin mini-prep kits can be purchased from QIAGEN, Inc. (Valencia, Calif.). DNA sequencing will be carried out by the UCLA DNA Sequencing Center (Los Angeles, Calif. USA) or the University of Florida DNA Sequencing Core Laboratory (Gainesville, Fla. USA). Purification of enzymes can be accomplished using Fast Flow DEAE-Sepharose (Pharmacia), CM-celullose (Whatman) or similar ionic exchange materials. Other key enzymes and reagents can be purchased from well-known vendors such as Sigma Chemical Company (St. Louis, Mo. USA), Aldrich Chemical Company (Milwaukee, Wis. USA), VWR (Pittsburgh, Pa. USA), and the like.

General Equipment to be Used:

Two SpectroMAX Plus plate readers (accepts both 96 and 384 well plates): Molecular Devices Corporation

Thermocycler for PCR: Perkin Elmer Model 9600

Deltacycler II System: Ericomp

Shaker/incubators: Lab-Line and New Brunswick Scientific

Gel Electrophoresis Apparatus: Bio-Rad and Pharmacia

Centrifuges: Eppendorf, Beckman, and Sorvall Model RC-3

Cell lysis: Branson Sonifier 250 and Avestin homogenizer

Lyophilizer (Aminco)

Gas Chromatograph (HP-5890)

HPLC system with diode array detector: Shimadzu VP series with autosampler

Robotic colony picker: Autogen

### Example 1

### Formate Dehydrogenase Mutants

Formate dehydrogenase mutants were prepared based on formate dehydrogenase having the following native protein sequence (SEQ ID 1):

MGKIVLVLYDAGKHAADEEKLYGCTENKLGIANWLKDQGHELITTSDKEG
ETSELDKHIPDADIIITTPFHPAYITKERLDKAKNLKLVVVAGVGSDHID
LDYINQTGKKISVLEVTGSNVVSVAEHVVMTMLVLVRNFVPAHEQIINHD
WEVAAIAKDAYDIEGKTIATIGAGRIGYRVLERLLPFNPKELLYYDYQAL
PKEAEEKVGARRVENIEELVAQADIVTVNAPLHAGTKGLINKELLSKFKK
GAWLVNTARGAICVAEDVAAALESGQLRGYGGDVWFPQPAPKDHPWRDMR
NKYGAGNAMTPHYSGTTLDAQTRYAEGTKNILESFFTGKFDYRPQDIILL
NGEYVTKAYGKHDKK.

Assays of the mutated FDH's were carried out as described above. The following data are specific activities with respect to FDH (corrected for % protein and % purity by PAGE). All of these activities are measured under saturating conditions (200 mM Formate, 10 mM NAD or NADP, pH 7.5, 100 mM KPO4, Room Temperature):

| | NAD Activity | NADP Activity |
|---|---|---|
| Enzyme | (U/mg FDH) | (U/mg FDH) |
| WT FDH | 2.2 | 0.0013 |
| FDH 1.3 | 1.5 | 0.083 |
| FDH 2.1 | 1.3 | 0.19 |
| FDH 3.1 | 1.3 | 0.36 |

The mutations are as follows:

| | |
|---|---|
| FDH 1.3 | D195S |
| FDH 2.1 | D195S, Y196H |
| FDH 3.1 | D195S, Y196H, K356T |

### Example 2

### Leucine Deydrogenase Mutants

Leucine dehydrogenase mutants were prepared based on leucine dehydrogenase having the following native protein sequence (SEQ ID 2):

MGKIFDYMEKYDYEQLVMCQDKESGLKAIICIHVTTLGPALGGMRMWTYA
SEEEAIEDALRLGRGMTYKNAAAGLNLGGGKTVIIGDPRKDKNEAMFRAL
GRFIQGLNGRYITAEDVGTTVEDMDIIHEETRYVTGVSPAFGSSGNPSPV
TAYGVYRGMKAAAKEAFGDDSLEGKVVAVQGVGHVAYELCKHLHNEGAKL
IVTDINKENADRAVQEFGAEFVHPDKIYDVECDIFAPCALGAIINDETIE
RLKCKVVAGSANNQLKEERHGKMLEEKGIVYAPDYVINAGGVINVADELL
GYNRERAMKKVEGIYDKILKVFEIAKRDGIPSYLAADRMAEERIEMMRKT
RSTFLQDQRNLINFNNK.

Four mutants were created and identified through screening that showed enhanced activity toward branched chain amino acids L-leucine, L-isoleucine, L-valine, and L-tert-leucine. The four mutations were as follows: F102S, V33A, S351T and N145S. Increases in activity were from 1.5 to 4 fold relative to the starting wild-type enzyme.

### Example 3

### Additional Leucine Deydrogenase Mutants

Through standard molecular biological techniques, all possible combinations of the four mutations identified in Example 2 can be created. These mutants can be screened against various substrates to establish their catalytic activity for reductive amination or deamination reactions. It is also foreseen that other mutations at these positions can be made and screened, and that any of these mutations, or combinations of these mutations, can be used in conjunction with various silent mutations in the gene.

### Example 4

### Galactose Oxidase Mutants

Galactose oxidase mutants were prepared based on galactose oxidase having the following native protein sequence (SEQ ID 3):

MASAPIGSAISRNNWAVTCDSAQSGNECNKAIDGNKDTFWHTFYGANGDP
KPPHTYTIDMKTTQNVNGLSMLPRQDGNQNGWIGRHEVYLSSDGTNWGSP
VASGSWFADSTTKYSNFETRPARYVRLVAITEANGQPWTSIAEINVFQAS
SYTAPQPGLGRWGPTIDLPIVPAAAAIEPTSGRVLMWSSYRNDAFGGSPG
GITLTSSWDPSTGIVSDRTVTVTKHDMFCPGISMDGNGQIVVTGGNDAKK
TSLYDSSSDSWIPGPDMQVARGYQSSATMSDGRVFTIGGSWSGGVFEKNG
EVYSPSSKTWTSLPNAKVNPMLTADKQGLYRSDNHAWLFGWKKGSVFQAG
PSTAMNWYYTSGSGDVKSAGKRQSNRGVAPDAMCGNAVMYDAVKGKILTF
GGSPDYQDSDATTNAHIITLGEPGTSPNTVFASNGLYFARTFHTSVVLPD
GSTFITGGQRRGIPFEDSTPVFTPEIYVPEQDTFYKQNPNSIVRVYHSIS
LLLPDGRVFNGGGGLCGDCTTNHFDAQIFTPNYLYNSNGNLATRPKITRT
STQSVKVGGRITISTDSSISKASLIRYGTATHTVNTDQRRIPLTLTNNGG
NSYSFQVPSDSGVALPGYWMLFVMNSAGVPSVASTIRVTQ.

By mutagenesis and screening against aryl alcohol substrates, the following mutants of galactose oxidase were created and identified by sequencing.

| Ref number | Mutation location |
|---|---|
| 98 | M278T, V492A, N535D |
| 110 | N521S, S567T |
| 112 | R217C, V494A |
| 146 | R217C, M278T, V492A, N535D |
| 158 | R217C, M278T, V492A, V494A, N535D |
| 163 | R217C, M278T, V492A, N521S, N535D |
| 164 | R217C, M278T, V492A, N535D, S567T |
| 165 | Q406L |
| 166 | M278T, Q406L, V492A, N535D, |
| 176 | R217C, M278T, V492A, V494A, N521S, N535D |
| 177 | R217C, M278T, Q406R, V492A, N535D |
| 178 | R217C, M278T, Q406R, V492A, N535D, T549I |
| 179 | T94A, R217C, M278T, Q406R, V492A, N535D |
| 180 | N25Y, R217C, M278T, V492A, N535D, T578S, |
| 185 | D216N, M278T, Y329C, Q406L, V492A, N535D |
| 186 | M278T, Y329C, Q406L, V492, N535D |
| 187 | R217C, M278T, Q406L, V492A, V494A, N521S, N535D |
| 202 | R217C, M278T, Q406Y, V492A, V494A, N521S, |
| | N535D, T578S |
| 203 | R217C, M278T, V492A, V494A, N521S, S, N535D, |
| | T578S |

The mutations listed in the table can all be prepared in various combinations by methods known to those skilled in the art, creating still additional unique mutants with enhanced aryl alcohol oxidase activity. All such mutants are envisioned herein and specifically claimed. The individual mutations which may be combined in all possible combinations are as follows: N25Y, T94A, D216N, R217C, M278T, Y329C, Q406R, Q406L, V492A, V494A, N521S, N535D, T5491, S567T, T578S. It is also foreseen that other mutations at these positions can be made and screened, and that any of these mutations, or combinations of these mutations, can be used in conjunction with various silent mutations in the gene.

The preceding description has been presented with references to presently preferred embodiments of the invention. Persons skilled in the art and technology to which this invention pertains will appreciate that alterations and changes in the described methods can be practiced without meaningfully departing from the principle, spirit and scope of this invention. Accordingly, the foregoing description should not be read as pertaining only to the precise methods described, but rather should be read as consistent with and as support for the following claims, which are to have their fullest and fairest scope.

1. An amino acid sequence that is a mutant of an enzyme selected from the group consisting of leucine dehydrogenase sequences as described in SEQ ID 2, formate dehydrogenase sequence as described in SEQ ID 1, galactose oxidase sequences as described in SEQ ID 3, and substantial equivalents thereof, wherein:
when the amino acid sequence is a mutant of a leucine dehydrogenase sequence as described in SEQ ID 2 or a substantial equivalent thereof, the amino acid sequence contains at least one mutation selected from the group consisting of F102S, V33A, S351T, N145S and like mutations in subsantially equivalent sequences;
when the amino acid sequence is a mutant of a formate dehydrogenase sequence as described in SEQ ID 1 or a substantial equivalent thereof, the amino acid sequence contains at least one mutation selected from the group consisting of D195S, Y196H, K356T and like mutations in subsantially equivalent sequences; and
when the amino acid sequence is a mutant of a galactose oxidase sequence as described in SEQ ID 3 or a substantial equivalent thereof, the amino acid sequence contains at least one mutation selected from the group consisting of N25Y, T94A, D216N, R217C, M278T, Y329C, Q406R, Q406L, V492A, V494A, N521S, N535D, T549I, S567T, T578S and like mutations in subsantially equivalent sequences.

2. An amino acid sequence according to claim 1, wherein the sequence is a mutant of a leucine dehydrogenase sequence as described in SEQ ID 2 or its substantial equivalent.

3. An amino acid sequence according to claim 1, wherein the mutant is a mutant of a leucine dehydrogenase sequence as described in SEQ ID 2.

4. An amino acid sequence according to claim 1, wherein the mutant is a mutant of a leucine dehydrogenase sequence that is at least 45% homologous to the sequence described in SEQ ID 2.

5. An amino acid sequence according to claim 1, wherein the mutant is a mutant of a leucine dehydrogenase sequence that is at least 70% homologous to the sequence described in SEQ ID 2.

6. An amino acid sequence according to claim 1, wherein the mutant is a mutant of a leucine dehydrogenase sequence that is at least 80% homologous to the sequence described in SEQ ID 2.

7. An amino acid sequence according to claim 1, wherein the mutant is a mutant of a leucine dehydrogenase sequence that is at least 95% homologous to the sequence described in SEQ ID 2.

8. A deoxyribonucleic acid molecule containing a DNA sequence encoding the amino acid sequence of claim 2.

9. An amino acid sequence according to claim 1, wherein the sequence is a mutant of a formate dehydrogenase sequence as described in SEQ ID 1, or its substantial equivalent.

10. An amino acid sequence according to claim 1, wherein the mutant is a mutant of a formate dehydrogenase sequence as described in SEQ ID 1.

11. An amino acid sequence according to claim 1, wherein the mutant is a mutant of a formate dehydrogenase sequence that is at least 80% homologous to the sequence described in SEQ ID 1.

12. An amino acid sequence according to claim 1, wherein the mutant is a mutant of a formate dehydrogenase sequence that is at least 95% homologous to the sequence described in SEQ ID 1.

13. A deoxyribonucleic acid molecule containing a DNA sequence encoding the amino acid sequence of claim 9.

14. An amino acid sequence according to claim 1, wherein the sequence is a mutant of a galactose oxidase sequence as described in SEQ ID 3, or its substantial equivalent.

15. An amino acid sequence according to claim 1, wherein the mutant is a mutant of a galactose oxidase sequence as described in SEQ ID 3.

16. An amino acid sequence according to claim 1, wherein the mutant is a mutant of a galactose oxidase sequence that is at least 80% homologous to the sequence described in SEQ ID 3.

17. An amino acid sequence according to claim 1, wherein the mutant is a mutant of a galactose oxidase sequence that is at least 95% homologous to the sequence described in SEQ ID 3.

18. A deoxyribonucleic acid molecule containing a DNA sequence encoding the amino acid sequence of claim 14.

19. A method for the production of an amino acid that comprises contacting a ketoacid with the amino acid sequence of claim 2 in the presence of a reduced nicotinamide cofactor and an ammonia source.

20. A method for the recycling of a nicotinamide cofactor that comprises contacting an oxidized nicotinamid cofactor with an amino acid sequence of claim 9 in the presence of a formate source.
